(19) United States
(12) Patent Application Publication
(10) Pub. No.: US 2008/0188449 A1
(43) Pub. Date: Aug. 7, 2008

(54) Treatment of Conditions Caused By Calcium Abnormalities

(75) Inventors: Craig M. Crews, New Haven, CT (US); Stephanie J. Quinn, Hope Valley, RI (US); Stefan Somlo, Westport, CT (US); Dayne Okuhara, Westford, MA (US)

Correspondence Address: WOLF GREENFIELD & SACKS, P.C., 600 ATLANTIC AVENUE, BOSTON, MA, 02210-2206, US

(73) Assignee: Yale University, New Haven, CT (US)

(21) Appl. No.: 11/667,696

(22) PCT Filed: Nov. 15, 2005
(86) PCT No.: PCT/US05/41476

§ 371 Date: Nov. 7, 2007

Related U.S. Application Data
(60) Provisional application No. 60/627,844, filed on Nov. 15, 2004.

(51) Int. Cl. A61K 31/585; A61P 13/12; C12N 5/02; C12Q 1/02

(52) U.S. Cl. 514/175; 435/375; 435/29

(57) ABSTRACT

In certain aspects, the invention relates to use of PKD2 agonists, such as triptolide and triptolide derivatives, to regulate calcium release. In other aspects, the invention relates to use of PKD2 agonists to treat or aid in the treatment of any condition in which a calcium channel, such as the gene product of PKD 1 and/or PKD2, is mutated; calcium signaling is abnormal; or both, such as polycystic kidney disease.

### RELATED APPLICATION

This application claims the benefit of the filing date of U.S. Provisional Application No. 60/627,844, entitled “Triptolide treats polycystic kidney disease,” by Craig M. Crews and Stephanie J. Quinn, filed Nov. 15, 2004. The entire contents and teachings of the referenced provisional application are incorporated herein by reference.

### FUNDING

This invention was made with government support under Grant Number A1 055914 awarded by National Institutes of Health. The government has certain rights in the invention.

### BACKGROUND OF THE INVENTION

ADPKD, Autosomal Dominant Polycystic Kidney Disease, is the leading genetic cause for end stage renal failure. Mutations in the gene product of PKD1 (polycystin-1), account for approximately 85% of all cases of ADPKD; the remaining 15% is attributed to mutations in the gene product of PKD2 (polycystin-2) (Igarashi and Somlo, 2002, J Am Soc Nephrol 13, 2384-2398). Disease progression is characterized by the inability of tubule epithelium to regulate calcium flux, which results in a loss of the fully differentiated state, increased proliferation and the formation of fluid-filled cysts in the kidney. Normal cell growth in the nephron is under the control of the mechanosensory function of the primary cilia, where both polycystin-1 and polycystin-2 co-localize. In response to urine flow, the cell's primary cilium bends and calcium enters the cell through polycystin-2 (Koulen, et al., 2002, Nat Cell Biol 4, 191-197) activating signaling pathways required for maintaining growth arrest (Nauli, et al., 2003, Nat Genet 33, 129-137). Aside from organ transplantation, there is currently no therapeutic intervention to either cure or slow the progression of this disease.

Thus, there is a need for developing novel therapeutic compositions and methods for ADPKD.

### SUMMARY OF THE INVENTION

Applicants have shown that triptolide, a natural product from a Chinese medicinal herb, Tripterygium wilfordii hook-f stimulates intracellular calcium release and that one of the proteins affected by triptolide is polycystin 2 (PKD2), a calcium channel that is mutated in polycystic kidney disease (PKD). Applicants have also demonstrated a calcium dependent effect on triptolide binding and function and that at different concentrations, triptolide either arrests cell growth or actively induces cell death via apoptosis. Further, Applicants have assessed the therapeutic efficacy of triptolide in a model for ADPKD, which is characterized by mutations in the gene product of PKD1 and/or PKD2, abnormal calcium influx and disregulated cell proliferation. Based at least in part on the results of that assessment, Applicants provide a novel method of regulating calcium influx, arresting cell growth and reducing or slowing cyst progression in conditions in which a calcium channel, such as the gene product of PKD1 or PKD2, is mutated and/or calcium signaling is abnormal, as well as therapeutic agents (drugs) and pharmaceutical compositions useful in the method.

In certain embodiments, the present invention provides a method of treating or aiding in the treatment of polycystic kidney disease (PKD) (e.g., ADPKD or ARPKD) in an individual in need thereof. Such method comprises administering to the individual a therapeutically effective amount of a PKD2 agonist. As described herein, a “PKD2 agonist” mimics or enhances PKD2 activities such as calcium signaling. Optionally, the PKD2 agonist binds to PKD2 or enhancing interaction between PKD1 and PKD2. A specific example of the PKD2 agonist is a triptolide-related compound. The term “triptolide-related compound,” as used herein, includes triptolide, triptolide prodrugs, and triptolide derivatives or analogs. Exemplary triptolide-related compounds include, but are not limited to, triptolide, a triptolide prodrug, and a triptolide derivative such as triol-tripolide, triptonide, 14-methyl-triptolide, 14-deoxy-14α-fluoro-triptolide, 5α-hydroxy triptolide, 19-methyl triptolide, and 18-deoxo-19-dehydro-18-benzoyloxy-19-benzoyl triptolide, and 14-acetyl-5,6-didehydro triptolide. Optionally, the method further comprises administering to said individual a second therapeutic agent for treating PKD, for example, an EGF receptor kinase inhibitor, a cyclooxygenase 2 (COX2) inhibitor, a vasopressin V₂ receptor inhibitor, a ligand of a peripheral-type benzodiazepine receptor (PTBR), a somatostatin analogue (e.g., octreotide), and pioglitazone.

In one embodiment, the PKD2 agonist (e.g., a triptolide-related compound) is administered prior to the development of symptomatic renal disease in the individual such that PKD is prevented. For example, the individual has been determined to be at risk of PKD as determined by family history, renal imaging study and/or genetic screening. In another embodiment, the PKD2 agonist (e.g., a triptolide-related compound) is administered when the individual exhibits symptomatic renal disease such that the disease progression is slowed or inhibited. Preferably, the individual is a mammal such as a human. In certain cases, the PKD2 agonist is administered to an individual in combination with a surgical therapy such as partial removal of a kidney or kidney transplant. Although not wishing to be bound by any particular mechanism or theory, it is believed that the triptolide-related compound regulates calcium signaling in kidney cyst tissues in the present method.

In certain embodiments, the present invention provides a method of treating a cystic disease in an individual in need thereof. Such method comprises administering to the individual a therapeutically effective amount of a PKD2 agonist in an amount sufficient to slow or inhibit growth of cyst cells. For example, the cystic disease includes, but is not limited to, breast cysts, bronchogenic cysts, choledochal cysts, colloidal cysts, congenital cysts, dental cysts, epidermoid inclusions, hepatic cysts, hydatid cysts, lung cysts, mediastinal cysts, ovarian cysts, periapical cysts, pericardial cysts, and polycystic kidney disease (PKD). A specific example of the PKD2 agonist is a triptolide-related compound. Preferably, the individual is a mammal such as a human.

In certain embodiments, the present invention provides a method of slowing or inhibiting cyst formation. Such method comprises contacting cyst cells with a PKD2 agonist in an amount sufficient to slow or inhibit the cyst formation. To illustrate, the cyst cells are kidney cyst cells present in or isolated from an individual having or at risk of developing PKD (e.g., ADPKD). Preferably, the cyst cells are mammalian cells (e.g., human cells). A specific example of the PKD2 agonist is a triptolide-related compound. Optionally, the PKD2 agonist regulates calcium signaling in cyst cells in the present method.

In certain embodiments, the present invention provides a method of regulating calcium influx in a cell expressing polycystin-1 (PKD1) or polycystin-2 (PKD2). Such method comprises contacting the cell with an effective amount of a PKD2 agonist. Optionally, the cell is a kidney cell, such as a kidney cell present in or isolated from an individual having or at risk of developing PKD (e.g., ADPKD). A specific example of the PKD2 agonist is a triptolide-related compound, which includes, but not limited to, triptolide, a triptolide prodrug, and a triptolide derivative such as triol-tripolide, triptonide, 14-methyl-triptolide, 14-deoxy-14α-fluoro-triptolide, 5α-hydroxy triptolide, 19-methyl triptolide, and 18-deoxo-19-dehydro-18-benzoyloxy-19-benzoyl triptolide, and 14-acetyl-5,6-didehydro triptolide.

In certain specific embodiments, the present invention relates to the use of a PKD2 agonist for treating or aiding in the treatment of any condition in which a calcium channel, such as the gene product of PKD1 and/or PKD2, is mutated; calcium signaling is abnormal; or both. Also described herein is the use of PKD2 agonists (e.g., triptolide-related compound) to arrest (decrease, partially or completely) cellular proliferation and/or attenuate (slow, prevent or reverse) cyst formation by restoring calcium signaling in cystic cells such as those in PKD. Specifically described herein is the ability of a PKD2 agonist to arrest cellular proliferation and attenuate overall cyst formation, in a murine model of polycystic kidney disease, by restoring calcium signaling in these cells.

In certain embodiments, this invention provides a method of treating or aiding in the treatment of a condition (referred to as a condition caused by calcium abnormality) in which a calcium channel (e.g., PKD2) is mutated and/or calcium signaling is abnormal. Such method comprises administering a PKD2 agonist (e.g., a triptolide-related compound) to an individual in need of such treatment. A PKD2 agonist is administered in sufficient quantity to correct (partially or completely) the calcium abnormality and restore (partially or completely) calcium signaling, thereby treating or aiding in the treatment of the condition caused by calcium abnormality. As described herein, the phrase “restoring calcium signaling” refers to bringing calcium signaling to a level which results in arrest of cell proliferation and attenuation of cyst formation. In specific embodiments, such condition is KPD (e.g., ADPKD). In one embodiment, a PKD2 agonist is administered to the individual in sufficient quantity to regulate intracellular calcium release, particularly to restore (partially or completely) intracellular calcium release. In a further specific embodiment, a PKD2 agonist is administered to an individual in whom mutation is present in the PKD1 gene, but not in the PKD2 gene, to regulate activity/function of the PKD2 gene and prevent the individual from developing PKD or limit the extent to which PKD occurs. In each embodiment, calcium signaling is restored to such an extent to result in arrest of cellular proliferation and/or attenuation of cyst formation. Additional examples of conditions caused by a calcium abnormality include, but are not limited to, MCKD (medullary cystic kidney disease), TSC (Tuberous sclerosis), nephronophthisis, and Bardet-Biedl syndrome.

In certain embodiments, one or more PKD2 agonists (e.g., triptolide-related compounds) may be administered to the individual by a variety of routes, for example, orally, topically, parenterally, intravaginally, systemically, intramuscularly, rectally or intravenously. In certain embodiments, a PKD2 agonist is formulated with a pharmaceutical carrier.

In certain embodiments, a PKD2 agonist (e.g., a triptolide-related compound) can be administered alone or in combination with each other and/or with a second agent or drug, such as an EGF receptor kinase inhibitor, a COX2 inhibitor, a vasopressin V₂ receptor inhibitor, a ligand of PTBR, a somatostatin analogue (e.g., octreotide), and pioglitazone for treating ADPKD. For example, triptolide, a precursor thereof (e.g., a prodrug) or a triptolide derivative can be administered to an individual in need of treatment, alone or in combination with each other (e.g., triptolide and a triptolide analogue) or with a second agent or drug (e.g., triptolide and an EGF receptor kinase inhibitor). The second agent can be administered with a PKD2 agonist either in the same formulation or in separate formulations, to enhance treatment. In these embodiments, the PKD2 agonist and the second agent can be administered at the same time (simultaneously) or at separate times (sequentially), provided that they are administered in such a manner and sufficiently close in time to have the desired effect.

Also the subject of this invention are pharmaceutical compositions useful for treating or aiding in the treatment of an individual for a condition in which there is disruption of a calcium channel function, such as the gene product of PKD1 and/or the gene product of PKD2, is mutated; calcium signaling is abnormal; or both. Such compositions comprise one or more PKD2 agonists. For example, the compositions of the present inventions are useful for treatment or aiding in the treatment of PKD (e.g., ADPKD) in an individual in need thereof.

In further embodiments, the present invention relates to use of a PKD2 agonist in the manufacture of medicament for the treatment of a cystic disease and use of a PKD2 agonist in the manufacture of medicament for the treatment of a condition caused by abnormal calcium signaling.

### BRIEF DESCRIPTION OF THE DRAWINGS

FIGS. 1A-1B show triptolide analogs and their structural dependence to compete for binding. (A) Structures of triptolide and the analogs used in this study. (B) HeLa cells were treated with [³H]-triptolide for one hour in all samples after the addition of 10 μM or 1 μM of triptolide, one of its analogs, or no competition also for one hour. Cells were washed, total cell lysates prepared, and samples were counted for bound [³H]-triptolide activity, n=3. CPM=Counts per minute by scintillation counting.

FIGS. 2A-2E show that triptolide binding is specific, membrane localized, and saturable. (A) HeLa cells were treated with [³H]-triptolide for one hour in all samples. Competition of the radioligand was assessed by the addition of 1 μM unlabeled triptolide (cold) for one hour either before or after [³H]-triptolide addition, n=3. CPM=Counts per minute by scintillation counting. (B) HeLa cells were labeled with [³H]-triptolide and cellular fractions were prepared. Binding was assessed as total CPM in the cytosolic (S-100), membrane (P-100), or insoluble cellular fractions. (C) HeLa cells were labeled with [³H]-triptolide for one hour followed by preparation of total cellular lysates and addition to DEAE anion exchange resin. The resin was washed in batch elutions of increasing salt concentration following removal of the flow-through (FT). Each eluant was subsequently counted for [³H]-triptolide activity, n=3. (D) [³H]-triptolide labeled HeLa cell P-100 fractions were run out on 8% reducing or native PAGE. Gel slices were removed utilizing molecular weight marker designations, crushed and eluted in water, and then counted for [³H]-triptolide activity by scintillation counting, n=2. (E) HeLa cells were pre-incubated with 2 μM unlabeled triptolide or DMSO followed by increasing nanomolar concentrations of [³H]-triptolide for 1 hour. Specific binding was measured from scintillation counting of cellular lysates and receptor saturation was achieved. Non-specific binding (NSB) is shown in inset and does not reach saturation.

FIGS. 3A-3C show that extracellular calcium regulates triptolide mediated binding and cell death induction. (A) HeLa cells were cultured in the presence or absence of calcium containing media for 16 hours before the addition of 30 nM [³H]-triptolide to assess binding affinity, n=3. CPM=Counts per minute. (B) HeLa cells were cultured in the presence (+Ca²⁺) or absence (−Ca²⁺) of calcium containing media over a time course of 72 hours to determine the rate of growth in each condition, n=3. (C) HeLa cells were cultured in the presence (+Ca²⁺) or absence (−Ca²⁺) of calcium containing media plus 100 nM triptolide over a time course of 72 hours. Cells were washed with PBS, photographed, and counted using trypan blue at 0, 24, 48, and 72 hours to assess viability. Results are representative of three separate experiments.

FIGS. 4A-4C show that buffering cytosolic calcium can temporarily rescue triptolide induced cell death. (A) HeLa cells were cultured in the presence of calcium containing media and transfected with one of the following constructs for 24 hours: GFP vector, NLS-parvalbumin (PV)-GFP, or NES-PV-GFP. Images were acquired by confocal microscopy (40×). (B) Normal cell growth was assessed with each transient transfection construct, n=3. (C) 100 nM triptolide was added to all transfected cells and viability was assessed after 24 hours, n=3.

FIGS. 5A-5B show that inhibition of NFκB transactivation is independent of the presence of calcium. (A) HeLa cells were transfected with a κB-luciferase construct for all experimental conditions for 24 hours before the addition of 15 ng/ml TNF-α±100 nM triptolide. Cells were grown in the presence or absence of calcium containing media for 16 hours before treatment and then harvested for the assay after 6 hours, n=4. (B) Cells were transfected with one of the following: GFP vector, NES-PV-GFP, or NLS-PV-GFP at the same time as the κB-luciferase construct and treated as described in (A), n=4.

FIGS. 6A-6B show that triptolide concentration differentially effects viability/growth or NFκB Inhibition. (A) HeLa were plated at an initial concentration of 5×10⁵ and allowed to grow±triptolide (10-100 nM) for 48 hours. Viable (adherent) cells were photographed under 25× brightfield microscopy and cell death was assessed by trypan dye exclusion. Results are representative of 3 separate experiments. (B) Following a transient transfection with the κB-luciferase reporter construct, HeLa cells were incubated with 15 ng/ml TNF-α±triptolide (10-100 nM) for a total of 6 hours before assessing reporter activity, n=4.

FIGS. 7A-7C show structural divergence of biological functions of triptolide analogs. All experiments were done with HeLa cells, where cell viability was measured 24 hours after the addition of triptolide or one of its analogs at concentration ranges of 0.1-10 μM. Cell viability was assessed by trypan dye exclusion and recorded as the % change in cell number from the time of triptolide or analog addition. Control cells were allowed to grow in media alone and represent a normal population doubling, n=3. NFκB Inhibition was assessed following transfection with the κB-luciferase reporter construct and treatment with triptolide or one of its analogs (0.1-10 μM) and TNF-α for 5 hours. Control represents transfected cells without TNF-α addition, n=4. (A) Triptolide (1). (B) Triol-Triptolide (2). (C) Triptonide (3).

FIGS. 8A-8E show that triptolide induces a polycystin-2 dependent calcium release in murine kidney epithelial cells. (A-C) Cells were loaded with Fluo-4 and assessed for calcium release by fluorescence intensity under perfusion flow before and after 100 nM triptolide addition. Cell lines tested included (A) Pkd1^{-/-} (B) Pkd2^{-/-} and (C) Re-expression (Rex) of Pkd2 in the Pkd2^{-/-} background. (D) The average change in fluorescence amplitude was calculated from baseline levels (n=44 or 66 for Pkd2^{-/-} or Pkd2 Rex). (E) Western blot analysis of polycystin-2 expression in each of the cell lines tested.

FIGS. 9A-9J show that Pkd1^{-/-} murine kidney epithelial cells undergo growth arrest and p21 upregulation upon triptolide treatment. (A-E) Pkd1^{-/-} cells were treated with 100 nM triptolide over a time course of 96 hours. Representative fields were photographed under brightfield microscopy (10×). (F) Pkd1^{-/-} triptolide treated cells after 96 hours showing a flattened morphology (40×). (G) Confocal microscopy of Pkd1^{-/-} cells for polycystin-2 immunofluorescent expression (FITC, 40×). (H) Western blot analysis of p21 and (I) active caspase-3 expression in Pkd1^{-/-} cells during a time course of 100 nM triptolide treatment. (J) Viable cells were counted by the method of trypan blue dye exclusion in Pkd1^{-/-} (Mean±SE, n=5) and Pkd2^{+/-} (n=8) cells over a time course with 100 nM triptolide addition.

FIGS. 10A-10N show that triptolide reduces cystic burden in a Pkd1^{-/-} murine model of polycystic kidney disease. (A-C) Representative kidneys from Pkd1^{-/-} pups treated with DMSO during gestation (E10.5-birth). Large cysts are present throughout the medulla and cortex (10× magnification). (D-F) Representative kidneys from Pkd1⁻⁻ pups treated with triptolide during gestation. (G) Pkd1^{+/+} kidney from a pup treated with DMSO or (H) triptolide. (I) Pkd1^{+/-} kidney treated with DMSO or (J) triptolide. (K-M) IHC staining of Pkd1^{-/-} kidneys for active caspase-3 expression: (K) secondary antibody negative control, (L) DMSO treated, (M) triptolide treated. (N) The percent of cyst burden in the kidney as determined by area in each of the Pkd1 genotypes (Mean±SE, Pkd1^{-/-, n=}19; Pkd1^{+/-} and Pkd1^{+/+}, n=10).

### DETAILED DESCRIPTION OF THE INVENTION

The present invention is based, at least in part, on the discovery that agonists of polycystin-2 (PKD2), such as triptolide and/or triptolide derivatives (e.g., analogs), are effective in slowing or inhibiting growth of kidney cyst cells and in regulating calcium signaling. As described in the working examples, a large-scale protein purification strategy was designed to facilitate the identity of putative triptolide binding protein(s). Following chromatographic protein fractionation, SDS-PAGE separation, and MALDI-MS analysis, a 110 kD band was identified as polycystin-2 and served as a potential biological target for triptolide activity. Applicants have demonstrated herein a calcium dependent effect on triptolide binding and function and that triptolide can arrest cell growth or induce apoptosis, depending on the concentration at which it is administered. Based on triptolide's ability to modulate cell growth or death, as based upon its anti-tumor effects, and a putative mechanistic function through polycystin-2 channel activity, Applicants assessed the therapeutic efficacy of triptolide in a model of ADPKD. Triptolide and triptolide derivatives are used as examples of PKD2 agonists which can regulate calcium influx, arrest cell growth, or reduce or slow cyst progression. One of ordinary skill in the art will readily recognize that other PKD2 agonists can be derived using the methods as described below.

### Therapeutic Compounds

In certain aspects, the present invention relates to one or more PKD2 agonists for various therapeutic applications. As described herein, a “PKD2 agonist” mimics or enhances PKD2 activities. PKD2 activities include, but are not limited to, a PKD2-mediated calcium signaling event such as PKD2-mediated calcium release in cells. For example, a PKD2 agonist may directly bind to a PKD2 protein or enhances interaction between PKD1 and PKD2. To illustrate, PKD2 agonists can be small organic molecules, proteins, antibodies, peptides, peptidomimetics, or nucleic acids.

In a specific embodiment of the present invention, a PKD2 agonist is a triptolide-related compound. As used herein, the term “triptolide-related compound” includes triptolide, triptolide prodrugs, and triptolide derivatives (e.g., analogs). Optionally, triptolide derivatives or prodrugs are capable of regulating calcium release in cells and/or binding to a calcium channel such as PKD1 or PKD2.

With regard to structure, a triptolide “derivative” includes a compound derived from triptolide via a modification which can include, for example: substitution of a hydrogen atom or hydroxyl group with hydroxyl, lower alkyl or alkenyl, lower acyl, lower alkoxy, lower alkyl amine, lower alkylthio, oxo (═O), or halogen; or conversion of a single bond to a double bond or to an epoxide. In this sense, “lower” preferably refers to C₁ to C₄, e.g., “lower alkyl” refers to methyl, ethyl, or linear or branched propyl or butyl. Preferred hydrogen atom substitutions include hydroxyl, methyl, acetyl (C(O)CH₃) and fluoro.

For example, triptolide-related compounds include triol-tripolide and triptonide. Other examples of triptolide derivatives and prodrugs include 14-methyl-triptolide, 14-deoxy-14α-fluoro-triptolide, 5α-hydroxy triptolide, 19-methyl triptolide, and 18-deoxo-19-dehydro-18-benzoyloxy-19-benzoyl triptolide, and 14-acetyl-5,6-didehydro triptolide, e.g., those described in U.S. Pat. Nos. 5,663,335, 5,962,516, 6,150,539, 6,458,537, 6,569,893, and 6,943,259 (each of these U.S. patents is hereby incorporated by reference in its entirety). The triptolide derivatives and prodrugs can be prepared from triptolide by methods such as those described therein.

In certain embodiments, any of the triptolide-related compounds having an ionizable group at physiological pH may be provided as a pharmaceutically acceptable salt. This term encompasses, for example, carboxylate salts having organic and inorganic cations, such as alkali and alkaline earth metal cations (for example, lithium, sodium, potassium, magnesium, barium and calcium); ammonium; or organic cations, for example, dibenzylammonium, benzylammonium, 2-hydroxyethylammonium, bis(2-hydroxyethyl) ammonium, phenylethylbenzylammonium, dibenzylethylenediammonium, and the like. Other suitable cations include the protonated forms of basic amino acids such as glycine, ornithine, histidine, phenylglycine, lysine, and arginine.

In certain embodiments, many of the triptolide-related compounds act as prodrugs, by converting in vivo to triptolide. Compounds which are expected to convert to triptolide in vivo by known mechanisms, such as hydrolysis of an ester (organic or inorganic), carbonate or carbamate to an alcohol, or ring opening or ring closure from or to an epoxide or lactone, are referred to herein as prodrugs of triptolide or triptolide prodrugs. Such compounds are typically designed with such conversion in mind. These include, for example, the triptolide prodrugs described in U.S. Pat. Nos. 5,663,335, 5,962,516, 6,150,539, 6,458,537, and 6,569,893, and Published PCT Application No. WO 2003/101951.

The present invention also contemplates further PKD2 agonists obtainable from the screening methods described as below.

### Drug Screening Assays

In certain embodiments, the present invention provides assays for identifying PKD2 agonists. Such PKD2 agonists may serve as therapeutic agents for various conditions, such as a cystic disease, cancer, or any condition caused by abnormal calcium signaling. In certain embodiments, agents of the invention specifically modulate PKD2 activities, for example, PKD2-mediated calcium release in cells. Optionally, a PKD2 agonist may directly bind to PKD2 or enhances interaction between PKD1 and PKD2. It is understood that PKD2 agonists include small organic molecules, proteins, antibodies, peptides, peptidomimetics, or nucleic acids.

In certain specific embodiments, the present invention contemplates a screening assay that identifies agents that enhance PKD2-mediated calcium release in a test cell (e.g., a cell expression PKD2). In certain cases, the present invention relates to a screening assay that identifies PKD2 binding agents. The parameters detected in a screening assay may be compared to a suitable control. A suitable control may be an assay run previously, in parallel or later that omits the test agent. A suitable control may also be an average of previous measurements in the absence of the test agent. In general, the components of a screening assay mixture may be added in any order consistent with the overall activity to be assessed, but certain variations may be preferred.

In certain embodiments of the invention, assay formats include those which approximate such conditions as formation of ligand/receptor complexes, protein/protein complexes, PKD2-mediated calcium release, and anti-cyst activity. In certain cases, the assays may involve purified proteins or cell lysates, as well as cell-based assays which utilize intact cells. For example, simple binding assays can also be used to detect agents which bind to PKD2. Other binding assays may be used to identify agents that regulate interaction between PKD1 and PKD2. Specific examples of such assays can be found in the working examples below.

In an exemplary binding assay, a test compound is contacted with a recombinant PKD2 protein. Detection and quantification of the test compound/PKD2 complex provides a means for determining the test compound's ability to bind to PKD2. In another exemplary binding assay, a test compound is contacted with a cell expressing PKD2. PKD2-mediated calcium release is measured in the cell in the presence of the test compound or in the absence of the test compound. If the test compound increases PKD2-medicated calcium release, the test compound is a PKD agonist. The efficacy of the compound can be assessed by generating dose response curves from data obtained using various concentrations of the test compound. Moreover, a control assay can also be performed to provide a baseline for comparison. For example, in the control assay, the formation of complexes is quantitated in the absence of the test compound.

In certain embodiments of the present invention, the test compounds in the screening assays can be any chemical (element, molecule, compound, drug), made synthetically, made by recombinant techniques or isolated from a natural source. For example, these compounds can be peptides, polypeptides, peptoids, sugars, hormones, or nucleic acid molecules (such as antisense or RNAi nucleic acid molecules). In addition, these compounds can be small molecules or molecules of greater complexity made by combinatorial chemistry, for example, and compiled into libraries. These libraries can comprise, for example, alcohols, alkyl halides, amines, amides, esters, aldehydes, ethers and other classes of organic compounds. These compounds can also be natural or genetically engineered products isolated from lysates or growth media of cells—bacterial, animal or plant—or can be the cell lysates or growth media themselves. Presentation of these compounds to a test system can be in either an isolated form or as mixtures of compounds, especially in initial screening steps.

In a further embodiment of the invention, a candidate agent is identified as a PKD2 agonist in an animal model. In another further embodiment, the identified PKD2 agonist can be further characterized in an animal model for its therapeutic efficacy. The animal models include mice, rats, rabbits, and monkeys, which can be nontransgenic (e.g., wildtype) or transgenic animals. For example, the effect of the agent may be assessed in an animal model for any number of effects, such as its ability to slow or inhibit cyst growth in the animal and its general toxicity to the animal. Specific examples of such animal models include PKD1 or PKD2 deficient mice as described below in the working examples.

### Pharmaceutical Compositions and Administration Methods

In certain embodiments of methods of the present invention, a PKD2 agonist is formulated with a pharmaceutically acceptable carrier. A PKD2 agonist can be administered alone or as a component of a pharmaceutical formulation. As described herein, the term “formulation” and “composition” are used interchangeably. A PKD2 agonist may be formulated for administration in any convenient way for use in human or veterinary medicine. In certain embodiments, a PKD2 agonist included in the pharmaceutical preparation may itself be active, or may be a prodrug. The term “prodrug” refers to compounds which, under physiological conditions, are converted into therapeutically active agents.

Formulations containing one or more PKD2 agonists (e.g., triptolide-related compounds) for use in the methods of the invention may take the form of solid, semi-solid, lyophilized powder, or liquid dosage forms, such as tablets, capsules, powders, sustained-release formulations, solutions, suspensions, emulsions, ointments, lotions, or aerosols, preferably in unit dosage forms suitable for simple administration of precise dosages. The compositions typically include a conventional pharmaceutical carrier or excipient and may additionally include other medicinal agents, carriers, or adjuvants.

Optionally, the composition will be about 0.5% to 75% by weight of a compound or compounds of the invention, with the remainder consisting of suitable pharmaceutical excipients. For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

Formulations of the PKD2 agonist include those suitable for oral/nasal, topical, parenteral, intravaginal and/or rectal administration. The formulations may be administered to a subject (individual) orally, transdermally or parenterally, e.g., by intravenous, subcutaneous, intraperitoneal, or intramuscular injection. For use in oral liquid preparation, the composition may be prepared as a solution, suspension, emulsion, or syrup, being supplied either in liquid form or a dried form suitable for hydration in water or normal saline. For parenteral administration, an injectable composition for parenteral administration will typically contain the PKD2 agonist in a suitable intravenous solution, such as sterile physiological salt solution. Liquid compositions can be prepared by dissolving or dispersing the PKD2 agonist (generally about 0.5% to about 20%) and optional pharmaceutical adjuvants in a pharmaceutically acceptable carrier, such as, for example, aqueous saline, aqueous dextrose, glycerol, or ethanol, to form a solution or suspension. Dosage forms for the topical or transdermal administration of the PKD2 agonist include powders, sprays, ointments, pastes, creams, lotions, gels, solutions, patches, and inhalants.

The PKD2 agonist may also be administered by inhalation, in the form of aerosol particles, either solid or liquid, preferably of respirable size. Such particles are sufficiently small to pass through the mouth and larynx upon inhalation and into the bronchi and alveoli of the lungs. In general, particles ranging from about 1 to 10 microns in size, and preferably less than about 5 microns in size, are respirable. Liquid compositions for inhalation comprise the active agent dispersed in an aqueous carrier, such as sterile pyrogen free saline solution or sterile pyrogen free water. If desired, the composition may be mixed with a propellant to assist in spraying the composition and forming an aerosol.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

Methods for preparing such dosage forms are known or will be apparent to those skilled in the art; for example, see Remington's Pharmaceutical Sciences (19th Ed., Williams & Wilkins, 1995). The composition to be administered will contain a quantity of the selected compound in an effective amount, for example for treating an ADPKD patient as described herein. To illustrate, for administration to human patients, a reasonable range of doses may be 0.1 to 20 mg, depending upon the activity of the derivative compared to that of triptolide. While i.v. administration is preferred in a clinical setting, other modes of administration, such as parenteral or oral, may also be used, with higher dosages typically used for oral administration.

### Therapeutic Applications

In certain embodiments, the present invention relates to administration of a PKD2 agonist (e.g., a triptolide-related compound) for the treatment of a condition caused by abnormal calcium signaling, in slowing or inhibiting cyst growth, and in regulating calcium release (influx) and calcium signaling in cells. In one specific example, the present invention provides a method of treating or aiding in the treatment of polycystic kidney disease (e.g., ADPKD). Treatments include, but are not limited to, administration of e.g., a pharmaceutical composition, and may be prophylactic therapy, preventative therapy, or curative therapy (e.g., performed subsequent to the initiation of a pathologic event).

Polycystic kidney disease (PKD) is a major cause of end stage renal disease in humans. PKD is characterized by severe dilations of collecting ducts and can be inherited as an autosomal dominant (AD) or autosomal recessive (AR) trait. In humans, ADPKD has a later onset and slower progression than ARPKD, which usually affects newborns or young children. ARPKD can cause massive bilateral enlargement of the kidneys. Most individuals surviving the neonatal period eventually develop renal failure.

The large number of genes showing abnormal expression in cystic kidneys from humans and rodents with PKD suggests that cellular processes associated with signal transduction, transcriptional regulation, and cell-cycle control are involved in cyst formation and that the cellular defect in PKD directly affects the regulation of epithelial differentiation. A model of cyst development has been proposed which involves an autocrine loop where cyst epithelial cells synthesize epidermal growth factor (EGF) which is secreted into cyst lumens activating EGF receptors leading to increased proliferation. The human ADPKD kidney has been shown to overexpress c-myc mRNA.

In one embodiment of the present invention, a PKD2 agonist (e.g., a triptolide-related compound) is administered prior to the development of symptomatic renal disease in the individual for preventing PKD such as ADPKD. For example, the individual has been determined to be at risk of PKD as determined by family history, renal imaging study and/or genetic screening.

There are a variety of ways to screen or diagnose PKD. First, given the genetic nature of the disease, a careful review of family history can be undertaken. This information typically is obtained through family medical records and from the subject through a patient questionnaire that requests specific information on the health history of his or her relatives. Second, renal imaging study has become a common diagnostic tool in PKD. The specific kinds of imaging that can be employed to determine the development of cysts include ultrasound, CT scan, MRI, as well as other imaging techniques. Finally, genetic screening may be employed. For example, recent work suggests cyst formation is initiated as a result of a random somatic mutation of the remaining normal PKD allele in patients with germline disruption. In ADPKD patients, mutations in one PKD allele (PKD1 or PKD2) have been found. For example, analysis of the PKD alleles in cystic cells from ADPKD patients has revealed a loss of heterozygosity (LOH) or intragenic mutations involving the non-affected PKD1 allele in approximately 20% of renal cysts. Useful techniques for probing changes in chromosomal DNA and mRNA transcripts include RFLP analysis, RT-PCR coupled with sequence analysis, and SNP identification.

In one specific embodiment of the present invention, the PKD2 agonist (e.g., a triptolide-related compound) is administered when the individual exhibits symptomatic renal disease for preventing or treating PKD. As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample. The term “treating” as used herein includes prophylaxis of the named condition or amelioration or elimination of the condition once it has been established.

In certain embodiments, the present invention provides combination or multiple therapies for a condition such as PKD. For example, a PKD2 agonist (e.g., a triptolide-related compound) may therefore be used in combination with other therapeutic agents. These additional therapeutic agents include, but are not limited to, antiviral agents, anticancer agents, and anti-inflammatory agents. In a specific embodiment, methods of the present invention comprises administering to an individual a therapeutically effective amount of a PKD2 agonist and a second therapeutic agent for treating PKD, such as an EGF receptor kinase inhibitor, a COX2 inhibitor, a vasopressin V₂ receptor inhibitor, a ligand of PTBR, a somatostatin analogue (e.g., octreotide), and pioglitazone. For example, triptolide, a precursor thereof (e.g., a prodrug) or a triptolide derivative can be administered to an individual in need of treatment, alone or in combination with each other (e.g., triptolide and a triptolide analogue) or with a second agent or drug (e.g., triptolide and an EGF receptor kinase inhibitor). The second agent can be administered with a PKD2 agonist either in the same formulation or in separate formulations, to enhance treatment. In these embodiments, the PKD2 agonist and the second agent can be administered at the same time (simultaneously) or at separate times (sequentially), provided that they are administered in such a manner and sufficiently close in time to have the desired effect.

In certain embodiments, methods of the present invention comprise administering a therapeutically effective amount of a PKD2 agonist. The phrase “therapeutically effective amount,” as used herein, refers to an amount which results in the decrease or inhibition of cell growth of target cells (e.g., those affected by abnormal calcium signaling). For example, a therapeutically effective amount of a PKD2 agonist slows or inhibits cyst growth.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Studies on Calcium Dependence Reveal Multiple Modes of Action for Triptolide

Triptolide, a diterpene triepoxide isolated from the traditional Chinese medicinal vine, Trypterygium wilfordii hook f., has been shown to induce rapid apoptosis in myriad cancer cell lines and inhibit NFκB transactivation. To understand further the general cellular mechanisms for this therapeutically relevant natural product, binding and biological activities were assessed. Studies showed that triptolide binding was saturable, reversible and primarily localized to cell membranes. Depletion of calcium enhanced overall binding while differentially modulating biological function. Furthermore, triptolide's structural moieties demonstrated variability in the regulation of cell death versus inhibition of NFκB transactivation. These results implicate triptolide in the manipulation of at least two distinct cellular pathways with differing requirements for calcium and effective triptolide concentration in order to elicit each particular biological function.

1. [³H]-Triptolide Binding is Reversible and Associates with Cell Membranes.

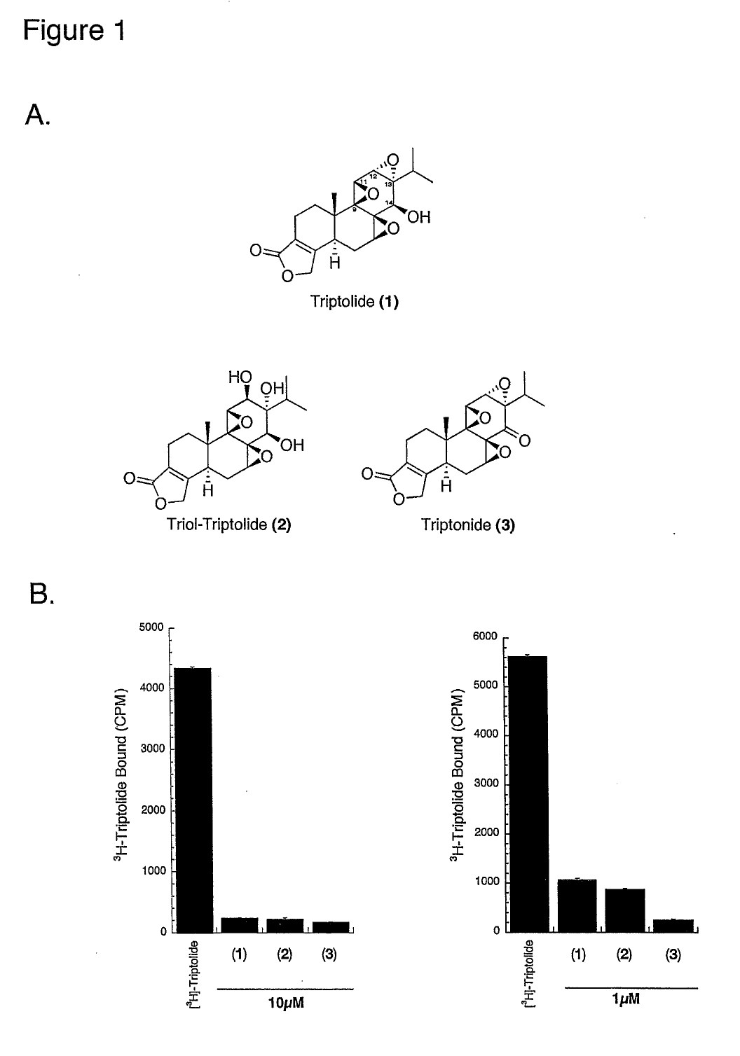

To gain insight into triptolide's mechanism of action Applicants sought to determine its specific binding activity utilizing a system employing [³H]-triptolide. In addition to competition with 1 μM or 10 μM unlabeled triptolide, Applicants also measured the binding affinities of triptolide analogs (FIG. 1A). These analogs have been previously described and differ structurally from triptolide in the hydrolysis of the 12,13 epoxide (2), and formation of the ketone at the C-14 hydroxyl (3) (FIG. 1A). HeLa cells readily bound [³H]-triptolide during an hour-long incubation and binding was significantly competed with pre-treatment of an excess of unlabeled triptolide (1-10 μM) (FIG. 1B). At either 1 or 10 μM, both triptolide analogs showed near total displacement of [³H]-triptolide (FIG. 1B). The effective interaction of these analogs with a presumed triptolide binding entity led us to their use in subsequent experiments addressing the mode of triptolide's biological functions.

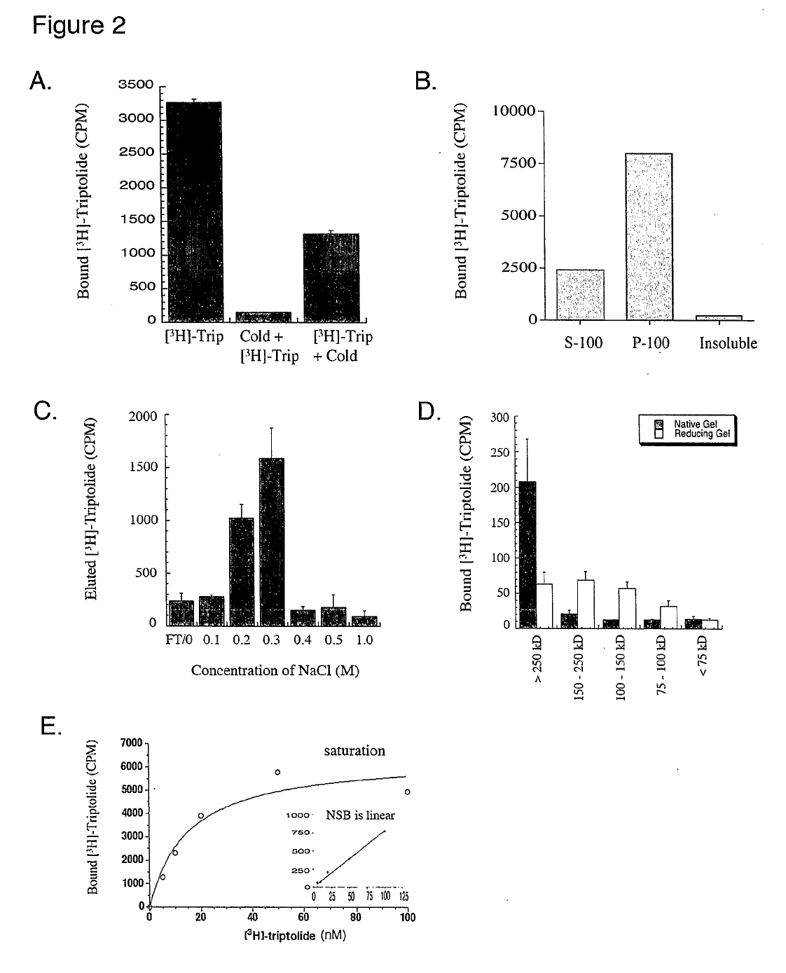

To address first the nature of triptolide binding within the cell, Applicants determined this interaction was reversible as [³H]-triptolide labeling was out competed by subsequent addition of excess unlabeled triptolide (FIG. 2A). An additional labeling experiment followed by cellular fractionation indicated that [³H]-triptolide binds predominantly to the membrane fraction (P-100) of the cell (FIG. 2B). Although 25% of the total cellular counts (i.e., representing bound triptolide) were found in the cytosolic fraction, it is uncertain whether this is specific binding or simply that free triptolide had dissociated from its binding protein due to experimental manipulation during fractionation. Since the data thus far had only shown binding within whole cells, Applicants next determined if this triptolide binding protein could be further characterized and/or enriched by its association with chromatographic reagents. HeLa cells were once again labeled with [³H]-triptolide and total cell lysates were then passed over the anion exchange resin DEAE. Batch elutions with increasing NaCl concentrations were used to disassociate interactions of variable charge. Substantial elution of [³H]-triptolide was not seen in the flow-through or with several salt-free washes and in fact did not begin to elute until the addition of 0.2 M NaCl (FIG. 2C). Additionally, free [³H]-triptolide diluted in lysis buffer and then passed over this resin eluted in the flow-through or during salt-free washes demonstrating that triptolide alone does not interact with this chromatographic media. Importantly, Applicants also observed that [³H]-triptolide would only bind to intact cells in culture but would not bind to any component of a total cellular lysate as assessed by interaction with the DEAE resin. Negative results for a triptolide binding interaction were also observed with DNA-cellulose and the cation exchange resin SPFF (sulphopropyl).

Another line of evidence for direct protein interaction involved a further separation of [³H]-triptolide labeled membrane preparations. Labeling of cells and total membrane purification by high speed centrifugation followed by detergent resolubilization yielded samples which were run out on 6% polyacrylamide gels under native or denaturing conditions (the [³H]-triptolide lysates were not boiled in either experimental condition). Gel slices were extracted based on molecular weight range, crushed, and water extracted followed by liquid scintillation. Native gel separation indicated that [³H]-triptolide was bound to protein(s) above 250 kD, while denaturing conditions showed a separation of [³H]-triptolide binding in a range from 75 to greater than 250 kD (FIG. 2D). These results suggested that triptolide could be binding to a protein complex that then dissociates upon reducing conditions. These data are also supported by size exclusion assays in which total cell lysates labeled with [³H]-triptolide retain the majority of binding interaction above a molecular weight cutoff of 100 kD.

Based upon the data above, Applicants next sought to determine if triptolide binding was saturable and if more than one protein (or binding site) was being targeted. Determination of the [³H]-triptolide binding affinity (K_{D}) and the binding capacity per cell (Bmax) were calculated using a saturation plot. HeLa cells were cultured to 90% confluency and subsequently treated with 0-100 nM of [³H]-triptolide for one hour. Non-specific binding was defined using 2 μM cold triptolide as competitor. Bmax was calculated to be 99±19 fmol triptolide bound/10⁵ cells of triptolide binding sites. Specific binding was found to be saturable with a K_{D} value of 15.5±0.8 nM, while non-specific binding was linear (non-saturable) (FIG. 2E).

### 2. Triptolide Binding Activity is Influenced by Extracellular Calcium Concentration.

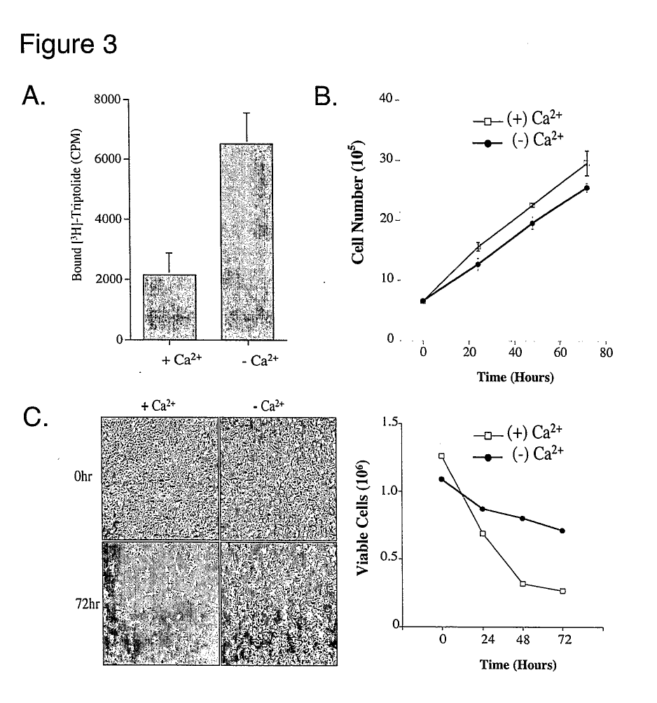

To understand further the nature of triptolide's interaction within the cell, Applicants altered cell culture conditions to examine if [³H]-triptolide binding would be affected. Calcium has been shown to mediate numerous cellular functions, including transcriptional activation of NF-AT and NFκB (Tomida, et al., 2003, EMBO J 22, 3825-3832; Dolmetsch, et al., 1997, Nature 386, 855-858; Dolmetsch, et al., 1998, Nature 392, 933-936). Additionally, it has been established that aberrant calcium signaling can result in cell death (Rizzuto, et al., 2003, Oncogene 22, 8619-8627; Orrenius, et al., 2003, Nat Rev Mol Cell Biol 4, 552-565). Due to triptolide's own link with NFκB and NF-AT and its propensity to induce cell death, Applicants examined if triptolide binding could be modulated by free calcium levels. Adherent HeLa cells were cultured in the presence or absence of calcium containing medium for 16 hours before [³H]-triptolide addition. Replicate experiments confirmed that in the absence of extracellular calcium, [³H]-triptolide binding significantly increased between 2-4 fold depending on cell number and density (FIG. 3A). It is also noteworthy that an increase in cell density (not cell number) also increases triptolide binding regardless of calcium levels. Additionally, specific calcium chelation by 10 mM EGTA for one hour in calcium containing medium increased binding by nearly 2 fold. These data indicate that triptolide interaction with its target protein(s) is potentially stabilized or enhanced when calcium levels are low.

### 3. Triptolide Induced Cell Death is Delayed in the Absence of Calcium.

Since extracellular calcium concentration can influence triptolide binding Applicants sought to determine if the presence of calcium influences the rate of triptolide-mediated apoptosis. To first establish the growth rate of HeLa in media±calcium, cell counts were performed over a 72 hour time course. Although calcium-free media caused cells to detach more easily, the overall growth rates were similar where cell doubling occurred every 24 hours on average (FIG. 3B). For triptolide experiments, cells were initially equilibrated in medium±calcium for 16 hours before the addition of 100 nM triptolide. Cell death was assessed by trypan blue dye exclusion at 24, 48, and 72 hours post drug treatment. In the presence of calcium-containing medium, triptolide induced at least 50% cell death by 24 hours with this trend continuing through later time points (FIG. 3C). In contrast, removal of calcium from the growth medium resulted in a higher proportion of viable cells (FIG. 3C). Following 72 hours of triptolide addition in calcium free media, only 35% of the cells had died indicating that there is a significant delay in this process. These results support a role for calcium in efficient cell death induced by triptolide. There is, however, a likely secondary (albeit slower) mechanism to promote apoptosis as the lack of calcium merely delays but does not eliminate cell death.

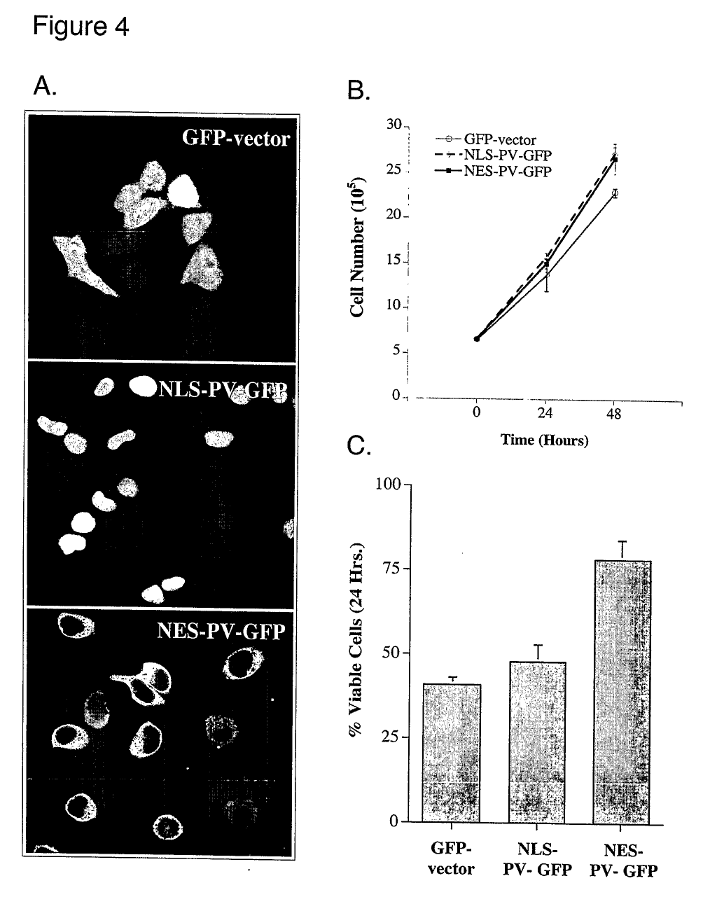

To investigate further the role of calcium in triptolide function Applicants utilized a system to buffer intracellular calcium levels. Various GFP-parvalbumin (PV) fusion proteins can be specifically localized to either the nucleus or the cytoplasm through a nuclear localization or exclusion signal (NLS or NES, respectively) (Pusl, et al., 2002, J Biol Chem 277, 27517-27527). Parvalbumin has two EF-hand calcium binding domains and can efficiently reduce the availability of free calcium in the cell (Pauls, et al., 1996, Biochim Biophys Acta 1306, 39-54). HeLa cells were transiently transfected with the GFP vector control, NES-PV-GFP, or NLS-PV-GFP in calcium containing media and efficient expression of each construct was determined by GFP localization (FIG. 4A). Normal cell growth was first assessed throughout 48 hours with each of the constructs. All transfections resulted in normal growth doubling during the course of the experiment without drug addition (FIG. 4B). For triptolide experiments, cells were transfected and allowed to express the construct for 24 hours before the addition of 100 nM triptolide. After 24 and 48 hours of treatment, cells were assessed for viability. Both control GFP vector and NLS-PV-GFP showed similar apoptosis induction whereby 50% of the cells were rounded and no longer viable after 24 hours (FIG. 4C). In marked contrast, cytosolic parvalbumin (NES-PV-GFP) significantly inhibited triptolide induced cell death at this time point (15-20% apoptosis) (FIG. 4C). This effect was transient however as there was complete cell death in all conditions by 48 hours. Since the parvalbumin buffering experiments are done in the presence of extracellular calcium, the cell can operate normally in that intracellular calcium stores may be refilled. It is reasonable to expect then if calcium homeostasis is being affected (i.e., cytosolic calcium levels increase) by triptolide, then parvalbumin would eventually reach a saturation point. This might explain why the rescue from apoptosis is transient through the 24 hour time point but is lost by 48 hours. These results not only confirm the overall importance of calcium to triptolide function, but more specifically point to cytosolic calcium levels as a mediator in triptolide induced cell death.

### 4. Inhibition of NFκB Transactivation by Triptolide is Calcium Independent.

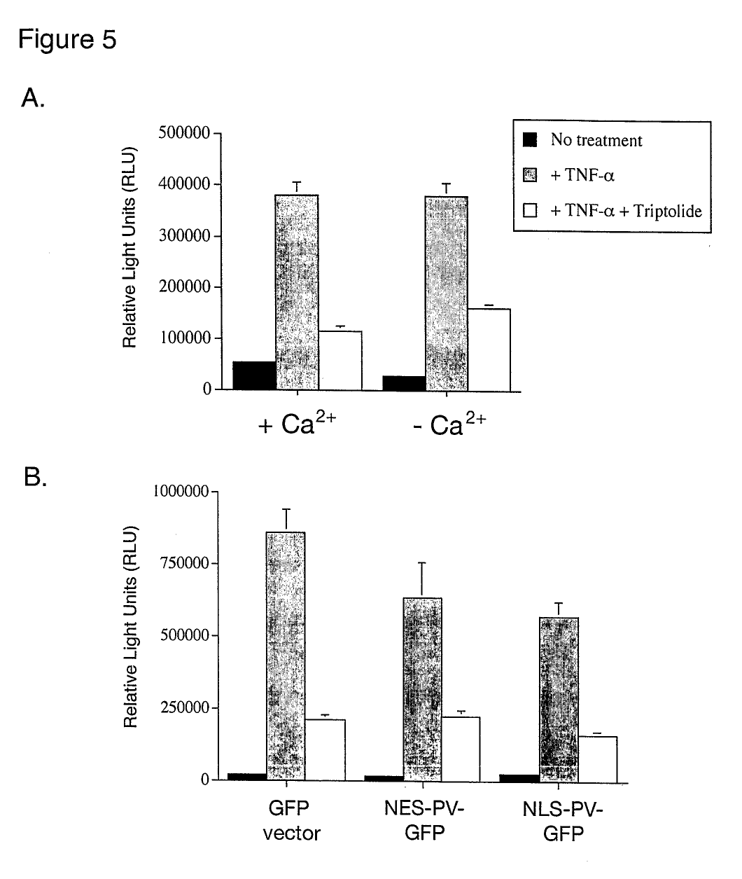

Having established that triptolide induced cell death is dependent upon free calcium concentration, Applicants next determined if this was also a requirement for the inhibition of NFκB transcription. HeLa cells were transiently transfected with the κB-luciferase reporter construct for 8 hours before being washed and then cultured in the presence or absence of calcium containing medium for 16 hours. Triptolide (100 nM) was pre-incubated with cells for 1 hour before addition of 15 ng/ml of TNF-α for 4 hours. Similar profiles were seen in both the presence and absence of calcium as TNF-α induced NFκB transactivation, while triptolide effectively inhibited it (FIG. 5A).

As an additional experiment, transcriptional activity was also assessed by site-directed calcium buffering. HeLa cells were co-transfected with the κB-luciferase plasmid as well as one of the following constructs: GFP empty vector, NES-PV-GFP, or NLS-PV-GFP and proper GFP localization was confirmed. Cells were grown in the presence of calcium for 24 hours before the addition of 100 nM triptolide and 15 ng/ml TNF-α. NFκB transactivation by TNF-α alone was quite high, although both NES- and NLS-parvalbumin transfected cells showed slightly lower levels of luciferase expression as compared to the vector control. Importantly, triptolide still retained the ability to suppress NFκB transactivation in all experimental conditions (FIG. 5B). These results suggest that while efficient induction of apoptosis by triptolide is calcium dependent, inhibition of NFκB transcriptional control is not.

### 5. Triptolide Concentration Differentially Effects Cell Death and Inhibition of NFκB.

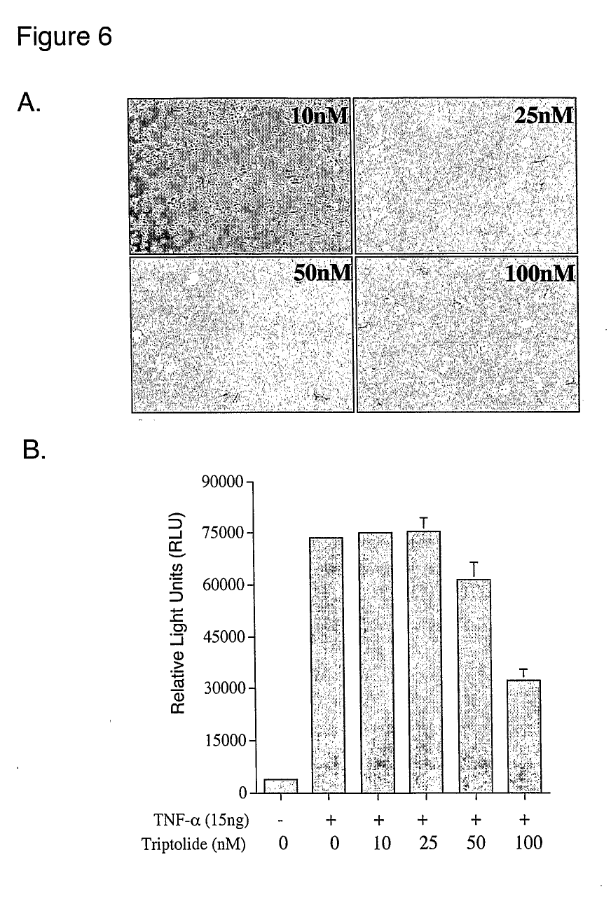

Applicants' results have implicated reversible binding of triptolide to a potential binding protein or complex that can be regulated by calcium. To understand if triptolide function is further separable between cell death and NFκB inhibition, Applicants examined the effect of concentration on these two endpoints. HeLa cells were cultured in the presence of 0, 10, 25, 50, or 100 nM of triptolide and separately assessed for cell death or the ability to suppress NFκB transactivation promoted by TNF-α. Following 24-48 hours of culture, viable cells were recovered and counted. Following 24 hours, triptolide concentrations from 25-100 nM caused greater than 50% of the cells to undergo cell death as assessed by detachment, clumping and the failure to exclude trypan blue dye. After 48 hours, nearly all cells treated within this concentration range of triptolide died (FIG. 6A). While untreated HeLa cells underwent two cycles of division, 10 μM triptolide inhibited cell proliferation but did not induce cell death. This is consistent with previous studies showing that low doses of triptolide cause cell cycle arrest rather than apoptosis (Kiviharju, et al., 2002, Clin Cancer Res 8, 2666-2674). It is also of note that triptolide's action on the cell that ultimately results in cell death is initially reversible, as three to four hours is the minimal incubation time required for commitment to apoptosis.

NFκB transactivation was examined using the κb-luciferase reporter construct. HeLa cells were transiently transfected and pre-treated with 0-100 nM triptolide for one hour prior to TNF-α addition. Cells were assessed for NFκB driven luciferase expression after an additional five hours of incubation, at which time TNF-α had induced transcriptional activity by approximately 15-fold in control cells. Both 10 and 25 nM triptolide did not inhibit TNF-α driven transcriptional activity of NFκB (concentrations shown to inhibit proliferation or induce cell death, respectively), whereas 50 nM suppressed activity by 20%, and 100 nM had the most profound effect with an average of 60% inhibition (FIG. 6B). It is also of note that luciferase activity assayed after 24 hours with 10 nM triptolide+TNF-α still showed greater than a 20-fold induction. Examination of these two biological endpoints at the same chronological time indicates that while 10 nM is efficient at arresting cell growth it cannot inhibit TNF-α induced NFκB transcriptional activity. The results thus far support a divergence of triptolide-mediated functions: the pathway regulating growth arrest/death is more sensitive to triptolide and calcium than the mechanism leading to transcriptional repression of NFκB.

### 6. Triptolide Analogs Show Differential Abilities to Induce Cell Death or Inhibit NFκB Transactivation.

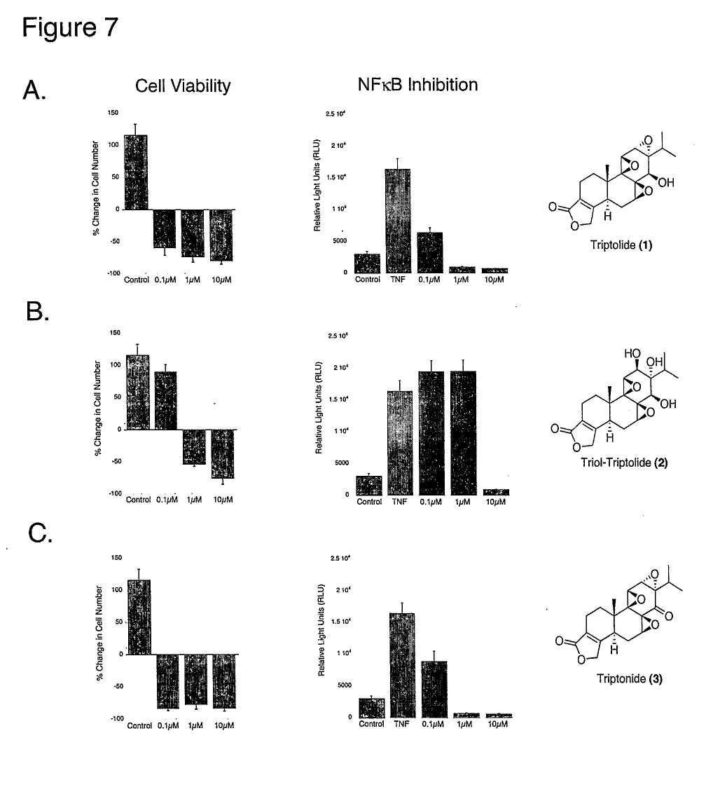

Based upon Applicants' studies examining the concentration dependent effect of triptolide on the two measured biological endpoints, cell death and transcriptional repression, Applicants wanted to determine how modulating triptolide's structure may also discriminate between the two pathways. For cell viability assays, HeLa cells were incubated with 0, 0.1, 1, or 10 μM of each analog or triptolide for 24 hours and then counted using trypan blue dye exclusion. Triptolide, as shown before, effectively induced greater than 50% cell death at 0.1 μM with no significant increase at the higher concentrations (FIG. 7A). NFκB transcriptional inhibition was measured using the κB-luciferase assay as previously described following 5 hours of incubation with each analog (0-10 μM) and TNF-α addition. NFκB inhibition was greater than 60% and attenuated further as triptolide's concentration increased to 1 or 10 μM (FIG. 7A).

Upon disruption of the 12,13 epoxide in analog (2), differential effects were seen in regards to each biological endpoint. At 0.1 μM analog (2), cell viability was not different from the untreated control (FIG. 7B), as compared to the 60% cell death observed at the equivalent concentration of triptolide.

However, if cells were allowed to continually grow at 0.1 μM analog (2) out to 72 hours, it became evident that there was an overall growth suppressive effect. Interestingly, 1 μM of analog (2), a concentration that efficiently competes with triptolide for binding (FIG. 1B) induced greater than 50% cell death while having no effect on NFκB transcriptional activity (FIG. 7B). It therefore appears that the mechanism of NFκB inhibition is more sensitive to the structural integrity of the 12,13 epoxide than is the cell growth/death regulatory pathway.

The most potent and biologically similar to triptolide was analog (3). Displacement of [³H]-triptolide binding was near complete at both the 1 and 10 μM concentration (FIG. 1B). In fact, 1 μM analog (3) actually elicited a higher competitive ability than triptolide itself at the same concentration (FIG. 1B). Both profiles of cell death and transcriptional repression mimicked triptolide with no significant difference from 0.1-10 μM (FIG. 7C). It is of note however that at 25 nM, a concentration shown to induce cell death by triptolide (FIG. 6A), analog (3) had only a growth suppressive effect. Since competition for binding by analog (3) was so strong, this data would support the idea that triptolide induced apoptosis at its lowest (25 nM) concentration is partially due to the functionality of the C-14 hydroxyl.

In sum, triptolide has a broad range of therapeutic potentials ranging from attenuation of inflammation, suppression of auto-immunity, and the elimination or regression of certain tumors. Basic studies of triptolide's mechanisms of action are incomplete with little understanding of how this small molecule can elicit such a broad range of effects. Utilizing [³H]-triptolide as a probe, Applicants have examined the properties of triptolide binding in the cell as well as addressed questions pertaining to two well described biological endpoints of triptolide function: cell death and transcriptional repression of NFκB. A specific triptolide binding activity is present within intact cells, is reversible, associates predominantly with cellular membranes, and is sensitive to calcium levels. While triptolide binding increases upon extracellular calcium depletion, it is severely impaired in its ability to induce cell death. This observed calcium dependence is specific to the regulation of apoptosis as triptolide's effect on NFκB transactivation is unaltered in the presence or absence of calcium. An overall separation of biological effects can be further discerned when triptolide is present at low nanomolar concentrations. While 10 nM is growth inhibitory and 25 nM induces cell death, neither of these concentrations can elicit transcriptional repression. Limited structure-function analysis utilizing triptolide analogs has demonstrated that while competitive binding for triptolide interaction sites is intact, biological effects are highly dependent upon structural moieties. These findings implicate triptolide as functioning through at least two separable pathways distinguishable by calcium requirements, sensitivity to drug concentration and preference towards structural entities. Further, Applicants have started to characterize a specific triptolide interaction within the cell so that triptolide-binding proteins may be identified in the future.

### 7. Experimental Procedures.

A) Reagents

Triptolide was obtained from Sinobest Inc. (China) and purity was 99% as determined by HPLC. DMSO was used to dissolve triptolide and was then directly added into culture media for all experiments. Triptolide was tritiated by Sib Tech, Inc. (Newington, Conn.) and resuspended in ethanol to a specific activity of 4-6 Ci/mmol. Purity was >95% as confirmed by RP-HPLC on a Hypersil C18 column and by TLC on both C18 and silica gel. Epi-Triptolide/Triol-Triptolide (C.A.S. No 147852-78-6), and Triptonide (C.A.S. No 38647-11-9) were purchased from Sequoia Research Products (United Kingdom).

B) Cell Culture and Viability Studies

HeLa cells were incubated in DMEM or SMEM (Gibco) media+10% FBS and maintained at 37° C. in 5% CO₂ for all experiments. HeLa cell viability was assessed by trypan blue dye exclusion, as well as by morphological examination (non-viable cells were rounded and detached from culture plate).

C) [³H]-Triptolide Labeling of HeLa Cells

Labeling studies were performed by the addition of approximately 30 nM [³H]-triptolide directly into the culture media for one hour at 37° C. For cold competition studies, 1 μM triptolide was incubated with the cells for one hour before or after the addition of [³H]-triptolide. Triptolide analog studies followed a similar protocol where concentrations used for competition were either 1 or 10 μM added before [³H]-triptolide. Medium was removed and cells were washed 3× in cold PBS. Total cell lysates were prepared (150 mM NaCl, 50 mM Tris-HCl pH 7.4, 1 mM EDTA, 1% Triton X-100, and Complete protease inhibitors (Roche)) and protein was quantitated before measuring the [³H]-triptolide binding activity via liquid scintillation.

DE-52 anion exchange resin (Whatman, Inc.), a diethylaminoethyl (DEAE)-cellulose, was prepared for binding by a 1 M sodium chloride (NaCl) wash followed by multiple washes with 0 M salt buffer (10 mM HEPES pH7.4, 0.1 mM EDTA, 1 mM DTT, 0.1% Triton X-100). HeLa cell lysates labeled with [³H]-triptolide were passed over the resin and allowed to bind at 4° C. for 30 minutes before collecting the flowthrough and subsequent washes. A step gradient from 0.0 to 1.0 M NaCl was used for protein elution. All fractions were subsequently counted by liquid scintillation.

For cellular fractionation studies, cells were allowed to swell on ice and lysed by passage through a syringe in a hypotonic lysis buffer (10 mM Tris-HCl+complete protease inhibitors). The lysate was centrifuged at 100,000×g and the supernatant was saved as the S-100 cytosolic fraction. The pellet was washed and resolubilized in 1% Triton X-100 containing lysis buffer. Following centrifugation, the supernatant was saved as the P-100 membrane fraction. Additionally, P-100 lysates were run out under native or reducing gel conditions without boiling. Gel slices were measured out in equal increments and the molecular weight range of each was calculated. Each gel piece was crushed in ddH₂O followed by scintillation counting of the water extract.

D) [³H]-Triptolide Specific Binding

Saturation binding assays were accomplished in HeLa cells adhered on 6-well plates in DMEM+10% FBS. All samples were at least 90% confluent at time of addition of triptolide. Non-specific binding of [³H]-triptolide was assessed by the pre-incubation of 2 μM (non-labeled) triptolide for one hour. Following cold competition (or DMSO) 5, 10, 20, 50, or 100 nM of [³H]-triptolide was added into the cultures for an additional one hour, and then cells were lysed and counted for binding activity.

E) Transfection of Parvalbumin Constructs

All parvalbumin-GFP constructs and control vectors (Pusl, et al., 2002, J Biol Chem 277, 27517-27527) were a gift of Anton Bennett (Yale University). Hela cells were plated out on 6- or 12-well plates at a density of 5×10⁵ or 1×10⁵, respectively. Cells were transiently transfected with 0.5-1 μg of one of the following pcDNA3 derived plasmids for 24 hours in DMEM/10% FBS+Lipofectamine 2000 (Invitrogen): CMV-parvalbumin-GFP, CMV-NES-parvalbumin-GFP, or CMV-NLS-parvalbumin-GFP. Following confirmation of GFP expression and localization by microscopy, 100 nM triptolide was added into each transfected cell population (>90% transfection efficiency). Cell viability was assessed at 24 and 48 hours post triptolide addition by morphology and trypan blue dye exclusion.

F) NF-Kappa B Luciferase Assay

A triple κB promoter-Luciferase reporter construct was a gift of Sankar Ghosh (Yale University). HeLa cells were plated at a density of 2×10⁵ in 12 well plates and transfected with 100 ng of the κB-Luciferase plasmid plus Lipofectamine 2000 (Invitrogen) for 24 hours before the addition of 100 nM triptolide for one hour and 15 ng/ml of recombinant (human) TNF-α (Roche) for an additional five hours. The transfection efficiency of HeLa was determined to be 80-90%, and all samples were normalized to protein concentration. Luciferase assays were performed using the Firefly luciferase kit as per manufacturer's protocol (Promega) and results obtained on the Wallac Victor2 1420 Multilabel Counter (Perkin Elmer).

### Example 2

### Triptolide Related Compounds Attenuate Polycystic Disease Progression Mediated by Polycystin-2

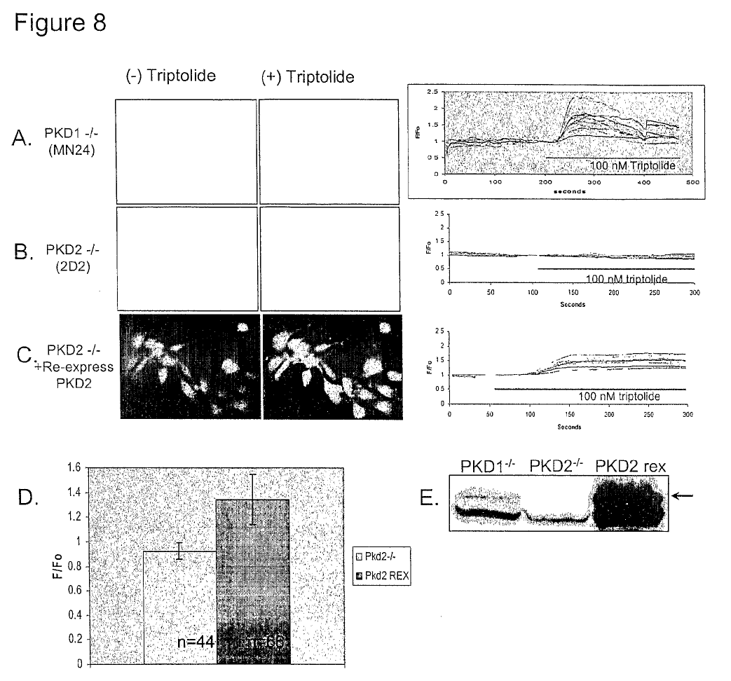

Murine kidney epithelial cell lines with differing polycystin-1 or polycystin-2 expression were used to establish a cellular based mechanism for polycystin-2 mediated calcium release in response to triptolide. Because the biochemical purification analysis identified polycystin-2 as a putative triptolide binding protein, Applicants assessed whether the calcium release was dependent upon expression of polycystin-1. Epithelial cells derived from the proximal nephric tubules of Pkd1^{-/-} mice were first examined to determine if calcium release was observed when 100 nM of triptolide was perfused through the imaging chamber. There was a clear rise in intracellular calcium levels upon triptolide addition, demonstrating that triptolide was capable of eliciting calcium release in cells (FIG. 8A); and furthermore, that this biological activity was not dependent upon polycystin-1 expression. When the identical system was used to perfuse 100 nM triptolide over Pkd2^{-/-} murine kidney epithelial cells, no calcium release was detected (FIG. 8B). To strengthen the evidence that polycystin-2 was necessary for calcium release elicited by triptolide addition, it was reconstituted by stable expression of Pkd2 into the background of Pkd2^{-/-} cells, which were assessed for sensitivity to triptolide. Re-expression of polycystin-2 restored calcium release in this cell line, providing mechanistic evidence for triptolide-mediated calcium regulation (FIG. 8C).

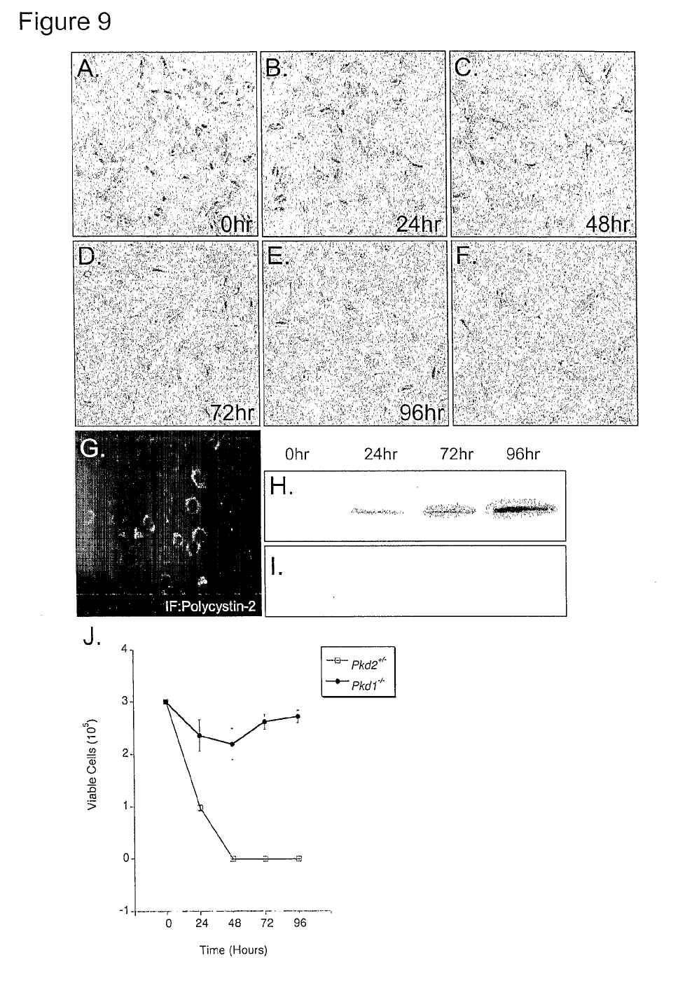

Calcium response to triptolide was, thus, shown to be dependent on polycystin-2. The biological response to calcium flux was next assessed in the murine Pkd1^{-/-} cell line, by adding 100 nM triptolide to the cultured cells and observing cell growth over time. Within the first 24 hours of culture, a minimal number of detached cells was observed. Over 96 hours, the remaining cells were growth arrested, as evidenced by their flattened morphology and the fact that the overall cell number did not increase (FIG. 9F). In the absence of triptolide, this cell line underwent a population doubling every 48 hours. In contrast, murine cell lines expressing at least one copy each of Pkd1 and Pkd2 (i.e., Pkd2^{+/-}) underwent rapid cell death within 24 hours, suggesting a more potent role for triptolide when both proteins are expressed and can associate (FIG. 9J). It is possible that additional signaling pathways are activated by triptolide when Pkd1 is expressed. PKD1^{-/-} cells have previously been shown to downregulate p21 expression as they proliferate (Bhunia, et al., 2002, Cell, 109:157-168). Therefore, Applicants assessed whether the inhibition of proliferation observed was due to p21 re-expression upon triptolide treatment. Over a 96 hour time course it became apparent that p21 was upregulated in the triptolide treated population, thereby re-establishing the normal state of growth arrest in these kidney epithelial cells (FIG. 9H). The presence of active caspase-3 was assessed by western blot analysis and again the results failed to implicate triptolide induced apoptosis in the Pkd1^{-/-} cell line (FIG. 9I). Thus, these in vitro data indicate that triptolide is capable of eliciting a polycystin-2 mediated calcium release, which results in p21 up-regulation and inhibition of Pkd1^{-/-} cell proliferation.

ADPKD is thought to result from a defect of calcium signaling due to the loss of the mechanosensory function of the primary cilia (Nauli, et al., 2003, Nat Genet 33, 129-137). Therefore, Applicants sought to establish if triptolide could artificially restore calcium flux in the Pkd1^{-/-} mouse model and arrest or delay the proliferative cystic state. Pkd1^{-/-} animals are not viable, although pups may develop to a late gestational state (E18.5-19.5). Such animals exhibit severe developmental abnormalities, such as cardiovascular (Boulter, et al., 2001, Proc Natl Acad Sci USA 98, 12174-12179; Kim, et al., 2000, Proc Natl Acad Sci USA 97, 1731-1736) and skeletal defects (Boulter, et al., 2001, Proc Natl Acad Sci USA 98, 12174-12179; Lu, et al., 2001, Hum Mol Genet 10, 2385-2396), in addition to kidney and pancreatic cyst formation (Wu, et al., 2002, Hum Mol Genet 11, 1845-1854; Lu, et al., 1997, Nat Genet 17, 179-181). Therefore, rescue from lethality seemed unlikely. Kidney cysts begin to form on E15.5 in the proximal tubules and rapidly progress into the cortex (Lu, et al., 1997, Nat Genet 17, 179-181). In Pkd1^{-/-} E18.5-19.5 pups, large kidney cysts are readily apparent upon gross morphological examination, as well as by histological staining.

Triptolide has been previously studied in rodent models of tumor regression (Tengchaisri, et al., 1998, Cancer Lett 133, 169-175; Yang, et al., 2003, Mol Cancer Ther 2, 65-72), but it had not yet been tested in a system utilizing pregnant females. To first establish a potential therapeutic versus lethal concentration of drug delivery, pregnant C57B1/6 mice were treated with incremental concentrations of triptolide between 0.01-0.15 mg/kg/day i.p. injections. Toxicity was assessed as determined by resorption of all embryos or the preponderance of a large percentage of stillborns. With reference to these criteria, triptolide toxicity was determined to be most prominent at concentrations of 0.1 mg/kg/day or greater. However, no discernable adverse effects were observed at a dosage of 0.07 mg/kg/day, which was used as the maximum tolerated dose. Another experimental parameter involved the timing of the start of triptolide injections, since polycystin-2 has been implicated in left-right axis formation in the developing embryo at approximately E7.75 (McGrath, et al., 2003, Cell 114, 61-73; Pennekamp, et al., 2002, Curr Biol 12, 938-943). Applicants therefore chose E10.5 as the start of triptolide injections, in order to allow for a normal polycystin-2 mediated patterning event and still leave sufficient time to act on cyst formation during kidney organogenesis.

Following successful Pkd1^{+/-}/Pkd1^{+/-} matings, 0.07 mg/kg/day of triptolide or DMSO control was injected i.p. into pregnant mice until they gave birth. All pups were assessed for viability, length, developmental staging, and wet kidney weight. A total of 59 pups from DMSO treated females and 100 pups from triptolide treated females were examined for multiple parameters, such as genotypic distribution, developmental stage at time of birth, and average kidney weights (Table 1). It has been previously demonstrated that Pkd1^{-/-} mice can be reabsorbed beginning at E12.5, due to the severe edema, vasculature defects and abnormal skeletogenesis, thereby resulting in an atypical Mendelian distribution of Pkd1^{-/-} progeny (Wu, et al., 2002, Hum Mol Genet 11, 1845-1854; Lu, et al., 2001, Hum Mol Genet 10, 2385-2396). The same reported deviation was observed in expected Pkd1^{-/-} numbers, with 20% and 18% for DMSO or triptolide treatment, respectively (Table 1). Approximately 20% of all Pkd1^{-/-} mice were born alive from each treatment group. However, severe edematous abnormalities were obvious upon necropsy. Independent of genotype, triptolide did not have any apparent overall deleterious effect on murine development or length of pregnancy.

TABLE 1
Descriptive Summaries for DMSO or Triptolide Treated Mice.

| | DMSO | 0.07 mg/kg/day Triptolide |
|---|---|---|
| Litters | 7 | 15 |
| Total Pups | 59 | 100* |
| Total Pkd1^{+/+} | 20 (34%) | 33 (33%) |
| Total Pkd1^{+/-} | 27 (46%) | 46 (46%) |
| Total Pkd1^{-/-} | 12 (20%) | 18 (18%) |
| Alive Pkd1^{-/-} (birth) | 2 (17%) | 4 (22%) |
| Ave. length Pkd1^{-/-} | 24.8 ± 0.4 | 24.6 ± 0.8 |
| (mm) | | |
| Ave. kidney wet | 8.0 ± 0.4 | 7.2 ± 0.2 |
| weight Pkd1^{+/+} (mg) | | |
| Ave. kidney wet | 7.5 ± 0.2 | 8.0 ± 0.3 |
| weight Pkd1^{+/-} (mg) | | |
| Ave. kidney wet | 17.5 ± 2.0 | 21.8 ± 2.8 |
| weight Pkd1^{-/-} (mg) | | |
| Ave. Delivery date | 19.4 ± 0.3 | 19.5 ± 0.3 |
| (Embryonic day) | | |

*3 are of unknown genotype, averages are presented as mean ± SE

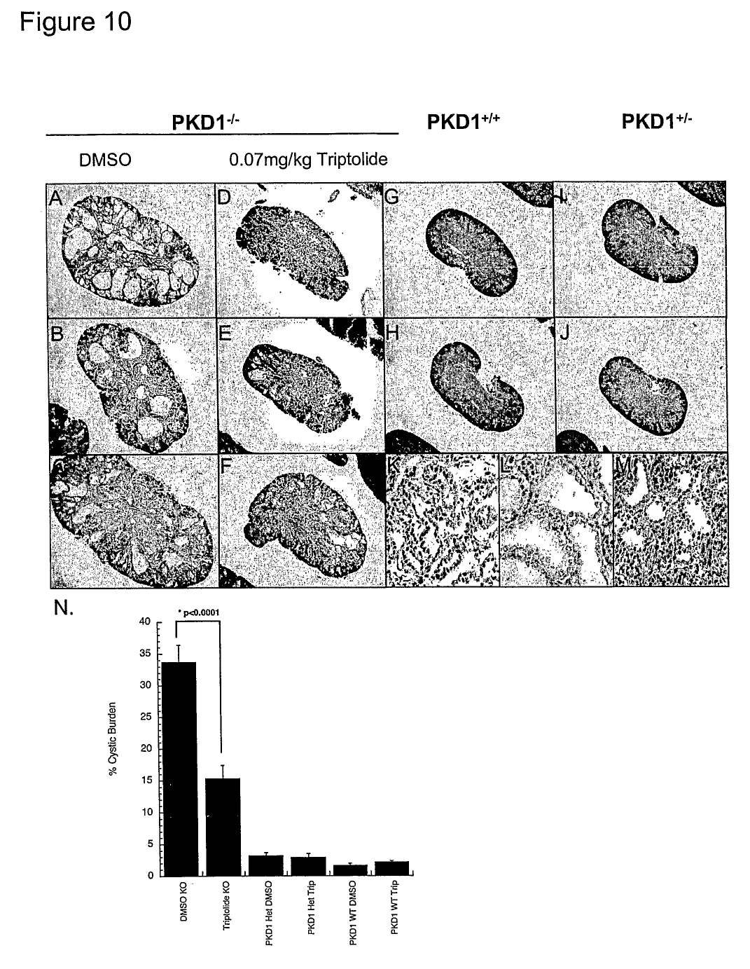

Initial examination of kidney pathology was by gross morphology. Pkd1^{-/-} kidneys, on average, were larger and in some cases cyst formation could be readily visualized. Wet weight kidney analysis from Pkd1^{+/+} or Pkd1^{+/-} mice demonstrated no significant difference in weight or overall size for DMSO or triptolide treated, respectively (Table 1). Pkd1^{-/-} kidneys were larger by weight, although there was no difference between DMSO or triptolide treatment (24.8±0.4 vs. 24.6±0.8 mg), indicating fluid secretion was not affected. Sagittal cross-sectioning of kidneys, H&E staining and the calculation of the area of cyst formation as a percentage of total kidney area was completed for each sample. Since the in vitro data have shown that the expression of both polycystin-1 and -2 results in cell death from triptolide treatment, it was possible that normal kidney development may have been adversely affected. This was not the case: Pkd1^{+/+} and Pkd1^{+/-} kidneys in both treatment groups showed normal morphology where background “cyst values” were calculated to account for random physiological abnormalities or artifacts of tissue handling and preparation. Pkd1^{-/-} kidneys from animals injected with DMSO had a mean cystic burden of 34±2.7%; several had cystic masses between of 55-65% of the whole kidney (FIG. 10A-C).

Triptolide treatment during the gestation of Pkd1^{-/-} pups resulted in a statistically significant decrease in the cystic burden to an average of 15±2.1% (FIG. 10D-F). There was some litter variability where there was a range from small kidneys with almost no evidence of any cyst formation, to a maximum cyst burden of 25%. This variability may be due to factors such as the proximity of triptolide delivery to the developing fetus during injections and difficulty in providing an effective therapeutic dose of triptolide while avoiding toxicity. The epithelial cells lining the cysts looked normal by microscopy and the diameter of cyst lumens on average was smaller. However, Applicants wanted to determine if the lack of cyst growth due to triptolide treatment was due to the induction of apoptosis or a delay in cell growth. To complement the in vitro data, tissue sections were stained for immunoreactivity towards active caspase-3, a marker of cellular commitment to apoptosis. Both DMSO (FIG. 10L) and triptolide (FIG. 10M) treated samples did not show any significant activation of the caspase pathway, as determined by comparison to secondary antibody staining alone (FIG. 10K). This is an indication the apoptotic pathway was not activated.

ADPKD cyst formation may be likened to benign epithelial neoplasia, in that both are characterized by uncontrolled cellular proliferation, independent of extracellular cues. Triptolide has been investigated for many of its potential therapeutic uses, including reduction of solid tumor masses, and is currently in clinical trials for its potent effect in a prostate cancer model (Kiviharju, et al., 2002, Clin Cancer Res 8, 2666-2674). In this respect, triptolide has been shown repeatedly to induce efficient apoptosis or cell growth arrest; the effect that results is dependent upon the effective concentration of the drug. Until now, upstream targets of triptolide efficacy have not been elucidated that explain its broad and potent biological effects. Furthermore, the discovery by our laboratory that polycystin-2 is required for triptolide mediated calcium release correlates with our previous findings that triptolide binding and cell death or growth arrest can be modulated by calcium concentration (see, e.g., Example 1).

Since ADPKD has no proven therapeutic cure or treatment, Applicants believe it is encouraging as a preliminary step to observe triptolide-mediated growth arrest and attenuation of cyst progression. Our animal model, while an excellent system to demonstrate PKD progression in neonatal development does limit the effective therapeutic concentration of triptolide that is permissive to the growing fetus. Future endeavors will allow for higher triptolide concentrations to be tested in older animals, as Applicants observed that greater than 0.15 mg/kg/day in an adult animal did not adversely affect its health. Additionally, although Applicants have demonstrated that triptolide reduces cyst progression in the absence of polycystin-1, it would be of future interest to establish if through an additional mechanism, triptolide could rescue the same phenotype in a polycystin-2 null model system. In summary, Applicants have established a novel pathway for triptolide mediated calcium release in a polycystin-2 dependent pathway that can reduce cystic burden in the kidneys of PKD mice. It is hopeful therefore that if fully developed, triptolide would be an ideal candidate for drug therapy as its history as an herbal therapy has already shown it to be well tolerated in humans.

### Materials and Methods

A) Cells and Reagents

The Pkd1^{-/-}(MN24), Pkd2^{+/-}(3B3) and Pkd2^{-/-}(2D2) murine cell lines were derived from knockout and transgenic mice as previously reported (Wu, et al., 1998, Cell, 93:177-88; Wu, et al., 2000, Nat. Genet, 24:75-8; Wu, et al., 2002, Hum Mol Genet, 11:1845-54). The PKD2-Rex cell line was made by stable integration of untagged PKD2 under hygromycin selection. Antibodies used included polycystin-2 (Cai, et al. 1999, J Biol Chem, 274:28557-65), cleaved (active) caspase-3 (Cell Signaling Technology) and p21 (BD Biosciences). Triptolide was obtained from Sinobest Inc. (China) and purity was 99% as determined by HPLC. DMSO was used to dissolve triptolide and was then directly added into culture media for all experiments. Triptolide was tritiated by Sib Tech, Inc. (Newington, Conn.) and resuspended in ethanol to a specific activity of 4-6 Ci/mmol. Purity was >95% as confirmed by RP-HPLC on a Hypersil C18 column and by TLC on both C18 and silica gel.

B) Calcium Imaging

Cells were plated on coverslips and loaded with Fluo-4 (Molecular Probes) diluted in DMSO/pluronic for 30 minutes prior to imaging. Cells were perfused with a calcium imaging buffer (HEPES, NaCl, KCl, MgSO₄ and CaCl₂)±100 nM triptolide. All cell traces are indicative of individual cellular fluorescence and calcium release. Data is presented as change of fluorescence over baseline control (no triptolide addition).

C) Immunoblotting and Immunofluorescence

Total cell lysates (0.5% Triton X-100, 50 mM Tris pH 7.4, 150 mM NaCl, 500 mM EDTA) were prepared for Western blot analysis and samples were run out by SDS-PAGE as per manufacturers' protocols. Brightfield images of cells were taken using a 10× or 40× objective. Confocal microscopy (40×) was used for immunofluorescence imaging of polycystin-2.

D) In Vivo Murine Experiments

As per approved IACUC animal protocol, Pkd1^{+/-}/Pkd1^{+/-} mice were mated and pregnant mice were divided into control (DMSO) or experimental (triptolide) groups. A total volume of 100 μl of PBS with no more than 5% DMSO or DMSO/0.07 mg/kg/day triptolide was injected i.p. with a 28 G ½ insulin syringe. Mice were weighed and injected starting at E10.5 until birth. All pups were examined for length and developmental staging such as whisker formation. Kidneys were harvested, weighed, and fixed in 4% paraformaldehyde before histological preparation.

E) Histological Examination

Kidneys were prepared by sagittal cross-sectioning and hematoxylin and eosin staining. All kidneys were photographed under the same magnification (4×) and cystic burden was computed using Image J analysis software (NIH). The area of cysts within the total area of the kidney (pixels) was calculated as a final percentage of cystic burden in the kidney. Immunohistochemical analysis of active caspase-3 was completed as per manufacturer's protocol.

F) Triptolide Binding Protein Purification

Five liters of HeLa-S cells (National Cell Culture Center) were labeled with a mixture of [³H]-triptolide as well as unlabelled triptolide for one hour at 37° C. Cells were harvested and washed 5× in cold PBS. The cell pellet was resuspended in hypolysis buffer (10 mM HEPES pH7.9, 10 mM KCl, 0.1 mM EDTA, Complete™ protease inhibitors (Roche), sodium orthovanadate, and DTT) and sheared through a syringe and needle. The supernatant was discarded and the pellet was resolubilized in lysis buffer containing 1% Triton X-100. The membrane fraction was subjected to further purification beginning with binding to the anion exchange resin DE-52 (Whatman). Final elution was completed with 0.3 M NaCl, and then passed through a size exclusion column with 100 kD cutoff (Amicon). The retentate was collected and bound to a Con A Sepharose (GE Healthcare) resin. The flow-through was collected and concentrated by passing over a 100 kD size exclusion column where the retentate was again collected and bound to Heparin Sepharose resin (GE Healthcare). Triptolide binding proteins were eluted with the addition of 1 M ammonium sulfate and 0.1% Triton X-100 and immediately bound to the hydrophobic resin Butyl Sepharose (GE Healthcare). Elution was performed using a no salt buffer (10 mM HEPES pH 7.4, 0.1 mM EDTA) with 1% triton X-100 and 2 mM EGTA. The eluant was subjected to a final concentration over a 100 kD size exclusion column followed by FPLC over a MonoQ anion exchange column. A step gradient of 0.0-1.0 M NaCl (10 mM HEPES pH 7.4, 0.1 mM EDTA, 0.5 M DTT) was run over the MonoQ column. 500 μl fractions were collected and the majority of [³H]-triptolide binding activity was observed between 0.3 M and 0.4 M NaCl. The corresponding fractions were concentrated and run out on by 8% SDS-PAGE and stained with Coomassie Blue. Bands were cut out from the gel and prepared for MALDI-TOF analysis. Proteins of interest were identified using Profound peptide mapping (Rockefeller University).

### INCORPORATION BY REFERENCE

All publications and patents mentioned herein are hereby incorporated by reference in their entirety as if each individual publication or patent was specifically and individually indicated to be incorporated by reference.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating or aiding in the treatment of polycystic kidney disease (PKD) in an individual in need thereof, comprising administering to the individual a therapeutically effective amount of a polycystin-2 (PKD2) agonist.

2. The method of claim 1, wherein the PKD2 agonist regulates PKD2-mediated calcium signaling in kidney cyst tissues.

3. The method of claim 1, wherein the PKD2 agonist is a small molecule.

4. The method of claim 1, wherein the PKD2 agonist is a triptolide-related compound.

5. The method of claim 4, wherein the triptolide-related compound is triptolide.

6. The method of claim 1, wherein the triptolide-related compound is a triptolide prodrug.

7. The method of claim 1, wherein the triptolide-related compound is a triptolide derivative selected from triol-tripolide, triptonide, 14-methyl-triptolide, 14-deoxy-14α-fluoro-triptolide, 50-hydroxy triptolide, 19-methyl triptolide, and 18-deoxo-19-dehydro-18-benzoyloxy-19-benzoyl triptolide, and 14-acetyl-5,6-didehydro triptolide.

8. The method of claim 1, further comprising administering to said individual a second therapeutic agent for treating PKD.

9. The method of claim 8, wherein the second therapeutic agent is selected from an EGF receptor kinase inhibitor, a cyclooxygenase 2 (COX2) inhibitor, a vasopressin V₂ receptor inhibitor, a ligand of a peripheral-type benzodiazepine receptor (PTBR), a somatostatin analogue (e.g., octreotide), and pioglitazone.

10. The method of claim 1, wherein the PKD2 agonist is administered prior to the development of symptomatic renal disease in the individual, whereby PKD is prevented.

11. The method of claim 10, wherein the individual has been determined to be at risk of PKD as determined by family history, renal imaging study and/or genetic screening.

12. The method of claim 1, wherein the PKD2 agonist is administered when the individual exhibits symptomatic renal disease, whereby the disease progression is slowed or halted.

13. The method of claim 1, wherein the PKD is ARPKD or ADPKD.

14. The method of claim 1, wherein the individual is a mammal.

15. The method of claim 14, wherein the individual is a human.

16. The method of claim 1, wherein the PKD2 agonist is administered by a route selected from oral administration, topical administration, parenteral administration, intravaginal administration, rectal administration, systemical administration, intramuscular administration, and intravenous administration.

17. The method of claim 1, wherein the PKD2 agonist is formulated with a pharmaceutically acceptable carrier.

18. A method of treating or aiding in the treatment of a condition caused by abnormal calcium signaling, comprising administering to an individual in need thereof a therapeutically effective amount of a PKD2 agonist.

19. The method of claim 18, wherein the abnormal calcium signaling is caused by reduced expression or activity of a calcium channel.

20. The method of claim 18, wherein the calcium channel is polycystin-2.

21. The method of claim 18, wherein the condition is PKD.

22. A method of treating a cystic disease in an individual in need thereof, comprising administering to the individual a therapeutically effective amount of a PKD2 agonist in an amount sufficient to slow or inhibit growth of cyst cells.

23. The method of claim 22, wherein the cystic disease is selected from breast cysts, bronchogenic cysts, choledochal cysts, colloidal cysts, congenital cysts, dental cysts, epidermoid inclusions, hepatic cysts, hydatid cysts, lung cysts, mediastinal cysts, ovarian cysts, periapical cysts, pericardial cysts, and polycystic kidney disease (PKD).

24. The method of claim 22, wherein the individual has or at risk of developing PKD.

25. A method of slowing or inhibiting cyst formation, comprising contacting cyst cells with a PKD2 agonist in an amount sufficient to slow or inhibit growth of cyst cells.

26. The method of claim 25, wherein the cyst cells are from an individual having or at risk of developing a cystic disease.

27. A method of regulating calcium influx in a cell expressing polycystin-2, comprising contacting the cell with an effective amount of a PKD agonist.

28. The method of claim 27, wherein the cell is a kidney cell.

29. The method of claim 27, wherein the kidney cell is from an individual having or at risk of developing PKD.

30. A method of identifying a PKD2 agonist, comprising:
(a) contacting a test agent to a cell expressing PKD2;
(b) measuring PKD2-mediated calcium release in the cell; and
(c) comparing the level of PKD2-mediated calcium release obtained in (b) with the level obtained in the absence of the test agent,
wherein a greater level of PKD2-mediated calcium release in the presence of the test agent than in the absence of the test agent indicates that the test agent is a PKD2 agonist.

31. The method of claim 30, wherein the cell is in an animal.

32. A method of identifying a therapeutic agent for slowing or inhibiting cyst formation, comprising:
(a) contacting a test agent to a cell expressing PKD2;
(b) measuring PKD2-mediated calcium release in the cell; and
(c) comparing the level of PKD2-mediated calcium release obtained in (b) with the level obtained in the absence of the test agent,
wherein a greater level of PKD2-mediated calcium release in the presence of the test agent than in the absence of the test agent indicates that the test agent is therapeutic agent for slowing or inhibiting cyst formation.

33. The method of claim 32, wherein the cell is in an animal.

34. Use of a PKD2 agonist in the manufacture of medicament for the treatment of a cystic disease.

35. Use of a PKD2 agonist in the manufacture of medicament for the treatment of a condition caused by abnormal calcium signaling.
